(19) United States
(12) Patent Application Publication
(10) Pub. No.: US 2020/0316364 A1
(43) Pub. Date: Oct. 8, 2020

(54) An Electrically Stimulated CBD Infused Pain-Relieving Patch

(71) Applicants: Quanqin Dai, Diamond Bar, CA (US); John Lasso, Lagina Hills, CA (US); Joseph Horton, Newport Beach, CA (US); Mark Julian, Newport Richie, FL (US); Trevor Theriot, Las Vegas, NV (US)

(72) Inventors: Quanqin Dai, Diamond Bar, CA (US); John Lasso, Lagina Hills, CA (US); Joseph Horton, Newport Beach, CA (US); Mark Julian, Newport Richie, FL (US); Trevor Theriot, Las Vegas, NV (US)

(21) Appl. No.: 16/377,217

(22) Filed: Apr. 7, 2019

(51) Int. Cl. A61N 1/04; A61B 5/00; A61N 1/36; A61K 31/352

(52) U.S. Cl. CPC A61N 1/0448; A61B 5/6833; A61K 9/7038; A61K 31/352; A61N 1/0456; A61N 1/36021

(57) ABSTRACT

An electrically stimulated CBD infused pain-relieving patch that is placed on a human's skin that is used to relieve pain when it is connected to a controlled electrical current generating device. The electrically stimulated CBD infused pain-relieving patch comprises of a carrier that has at least one electrode, at least one conductive electrode sheet that is attached to the carrier, and a conductive gel that is placed on the electrode sheet. The conductive gel contains at least one analgesic that is selected from the group consisting of Menthol, CBD, Lidocaine or Capsaicin. The conductive gel has inactive ingredients.

### CROSS REFERENCE

This application is a continuation application of U.S. patent application Ser. No. 16/375,816, filed on 4, Apr. 2019, titled An Electrically Stimulated CBD Infused Patch, which was a continuation-in-part of U.S. patent application Ser. No. 15/832,932, filed 6 Dec. 2017, titled Conductive Pad for pain relieving and muscle training.

### BACKGROUND

The present invention is directed to an electrically stimulated CBD infused pain-relieving patch that is placed on a human's skin.

A transdermal patch is a medicated adhesive patch that is placed on the skin to deliver a specific dose of medication through the skin and into the bloodstream.¹ ¹Available at https:en.wikipedia.org/wiki/Transdermal_patch, Wikipedia, Transdermal Patch, seen on Apr. 2, 2019.

Cannabidiol (hereinafter “CBD”) is a phytocannabinoid derived from Cannabis species, which is devoid of psychoactive activity, with analgesic, anti-inflammatory, antineoplastic and chemopreventive activities.² ²Available at https://pubchem.ncbi.nlm.nih.gov/compound/cannabidiol, National Center for Biotechnology Information. PubChem Database. Cannabidiol, CID=644019, https://pubchem.ncbi.nlm.nih.gov/compound/644019 (accessed on Apr. 3, 2019)

Capsaicin is a neuropeptide releasing agent selective for primary sensory peripheral neurons. Used topically, capsaicin aids in controlling peripheral nerve pain.³ ³Available at https://pubchem.ncbi.blm.nih.gov/compound/Capsaicin, National Center for Biotechnology Information. PubChem Database. Capsaicin, CID=1548943, https://pubchem.ncbi.nlm.nih.gov/compound/1548943 (accessed on Apr. 3, 2019).

Lidocaine patches are used to relieve the pain of post-herpetic neuralgia (PHN; the burning, stabbing pains, or aches that may last for months or years after a shingles infection).⁴ Lidocaine is a local anesthetic.⁵ It stops nerves from sending pain signals to the brain.⁶ ⁴Available at https://mediineplus.gov/druginfo/meds/a603026.html, US National Library of Medicine, Lidocaine Transdermal Patch, seen on Apr. 2, 2019.⁵id.⁶id.

Topical menthol gels are classified “topical analgesics” and are claimed to relieve minor aches and pains of the musculoskeletal system.⁷ Menthol has been reported to be effective in relieving pain with mild to moderate muscle strains.⁸ ⁷Available at https://www.ncbi.nlm.nih.gov/pmc/articles/PMC4178917/, Sundstrup E, Jakobsen M D, Brandt M, et al. Acute effect of topical menthol on chronic pain in slaughterhouse workers with carpal tunnel syndrome: triple-blind, randomized placebo-controlled trial. Rehabil Res Pract. 2014; 2014; 310913. doi:10.1155/2014/310913.⁸Available at Higashi Y, Kiuchi T, Furuta K, Efficacy and safety profile of a topical methyl salicylate and menthol patch in adult patients with mild to moderate muscle strain: a randomized, double-blind, parallel-group, placebo-controlled, multicenter study. Clinical Therapy 32. 2010; (1):34-43.

The present invention is a transdermal electrically stimulated patch (the patch has an analgesic gel impregnated on the patch) that is placed on a human's skin to alleviate pain. In use, the patch is adhered to the skin of a user and an electric current is applied to the patch via a controlled electrical generating device that attaches to the patch. The user's skin is stimulated by the current and the user's blood circulation is increased. Increased blood circulation allows the skin to absorb more of the analgesic impregnated on the transdermal patch by a factor of 12.

For the foregoing reason there is a need for an electrically stimulated CBD infused pain-relieving patch that is placed on a human's skin that is used to relieve pain.

### SUMMARY

The present invention describes an electrically stimulated CBD infused pain-relieving patch that is placed on a human's skin that is used to relieve pain.

The electrically stimulated CBD infused pain-relieving patch comprises of a transdermal patch that has electrodes that receive an electric current from a controlled electrical current generating device. The Pain-relieving patch has a conductive gel that is impregnated with at-least one analgesic that is selected from the group consisting of a Menthol, a CBD, a Lidocaine or a Capsaicin. The conductive gel also includes a plurality of inactive ingredients.

An object of the present invention is to provide an electrically stimulated CBD infused pain-relieving patch that will maximize the release of a pain reliever onto a user's skin.

Another object of the present invention is to provide an electrically stimulated CBD infused pain-relieving patch that will treat pain.

Yet another object of the present invention is to provide an electrically stimulated CBD infused pain-relieving patch that will increase blood flow in treated areas.

Still another object of the present invention is to provide an electrically stimulated CBD infused pain-relieving patch that will relieve pain associated with arthritis.

### DRAWINGS

These and other features, aspects, and advantages of the present invention will become better understood with regards to the following description, appended claims, and drawings where:

FIG. 1 a photograph that shows an electrically stimulated CBD infused pain-relieving patch system that is placed on a human's skin that is used to relieve pain;

FIG. 2 a photograph that shows a TENS device connected to the pain-relieving patch of the present invention: and

FIG. 3 is a view of an embodiment of the present invention.

### DETAILED DESCRIPTION

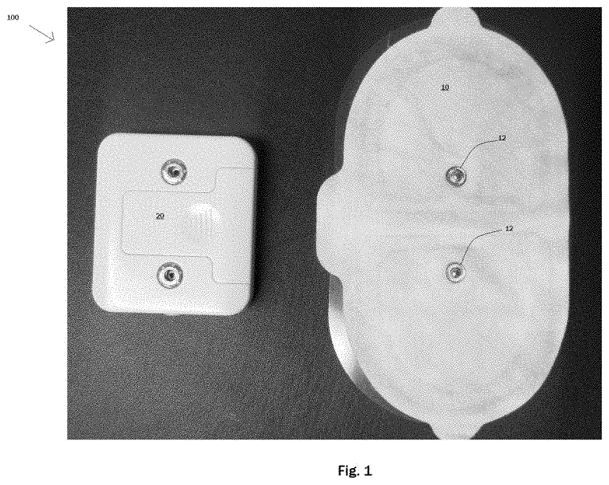

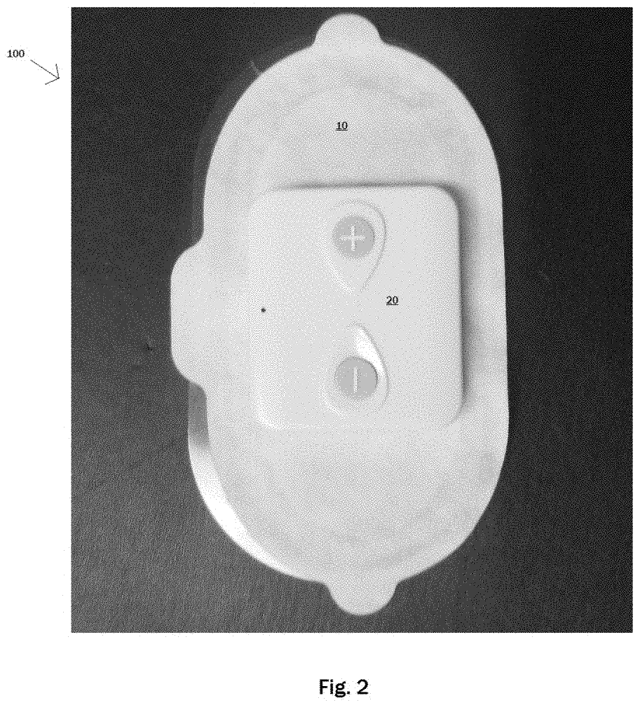

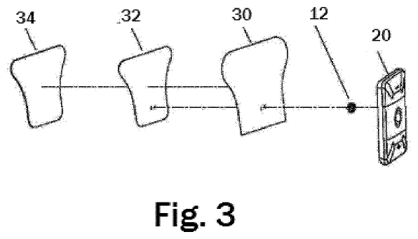

As seen in FIGS. 1-3, the present invention describes an electrically stimulated CBD infused pain-relieving patch 10 that is placed on a human's skin that is used to relieve pain. The pain-relieving patch 10 comprises of a carrier 30 that has at least one electrode 12, at least one conductive electrode sheet 32 that is attached to the carrier 30, and a conductive gel 34 that is placed on the electrode sheet 32. In use, the pain-relieving patch 10 is connected to a controlled electrical current generating device 20. The device can be either a TENS device, an interferential device or an EMS device.

The conductive gel 34 has at least one analgesic that is selected from the group consisting of a Menthol, a CBD, a Lidocaine or a Capsaicin. In a preferred embodiment, the at least one analgesic comprises of the CBD in the range from about 0.001 percent to about 20.000 percent of the gel and the Lidocaine in the range from about 0.001 percent to about 10.000 percent of the gel.

In another embodiment of the present invention, the at least one analgesic further comprises of the Menthol in the range from about 0.010 percent to about 20.000 percent of the gel.

In still another embodiment of the present invention, the at least one analgesic shall further comprise of the Capsaicin in the range from about 0.001 percent to about 8.000 percent of the gel.

The conductive gel of the present invention shall comprise of a plurality of inactive compounds. In a preferred embodiment, the plurality of inactive compounds shall be Water, Glycerin, Propylene Glycol, PVP, Sodium Polyacrylate, Methylparaben, Propylparaben, Aluminum Glycinate, Kaolin, Polyacrylic Acid, Polysorbate 80, Tartaric Acid, and/or Titanium Dioxide. Depending on the embodiment of the present invention, the inactive compounds shall be added to the conductive gel until a one hundred percent measurement is reached.

The patch is used by placing the patch on a user, connecting the patch to the controlled electrical generating device, powering the electrical generating device to deliver a charge. The pain-relieving patch can remain on a user's skin for a period of between eight to twelve hours. An Electrical Muscle Stimulation device can also be used to provide the electric current to the pain-relieving patch.

An advantage of the present invention is that it provides an electrically stimulated CBD infused pain-relieving patch that maximizes the release of a pain reliever onto a user's skin.

Another advantage of the present invention is that it provides an electrically stimulated CBD infused pain-relieving patch that treats pain.

Yet another advantage of the present invention is that it provides an electrically stimulated CBD infused pain-relieving patch that increases blood flow in treated areas.

Still another advantage of the present invention is that it provides an electrically stimulated CBD infused pain-relieving patch that relieves pain associated with arthritis.

While the inventor's above description contains many specificities, these should not be construed as limitations on the scope, but rather as an exemplification of several preferred embodiments thereof. Accordingly, the scope should not be determined by the embodiments illustrated, but by the appended claims and their legal equivalents.

What is claimed is:

1. An electrically stimulated CBD infused pain-relieving patch that is placed on a human's skin that is used to relieve pain when it is connected to a controlled electrical current generating device, the patch comprises of a carrier that has at least one electrode, at least one conductive electrode sheet that is attached to the carrier, and a conductive gel that is placed on the electrode sheet.

2. The electrically stimulated CBD infused pain-relieving patch that is placed on a human's skin that is used to relieve pain when it is connected to a controlled electrical current generating device of claim 2, wherein the conductive gel is comprised of at least one analgesic that is selected from the group consisting of a Menthol, a CBD, a Lidocaine or a Capsaicin.

3. The electrically stimulated CBD infused pain-relieving patch that is placed on a human's skin that is used to relieve pain when it is connected to a controlled electrical current generating device of claim 2, wherein the at least one analgesic comprises of the CBD that is in the range from about 0.001 percent to about 20.000 percent of the gel and the Lidocaine that is in the range from about 0.001 percent to about 10.000 percent of the gel.

4. The electrically stimulated CBD infused pain-relieving patch that is placed on a human's skin that is used to relieve pain when it is connected to a controlled electrical current generating device of claim 3, wherein the at least one analgesic comprises of the Menthol that is in the range from about 0.010 percent to about 20.000 percent of the gel.

5. The electrically stimulated CBD infused pain-relieving patch that is placed on a human's skin that is used to relieve pain when it is connected to a controlled electrical current generating device of claim 4, wherein the at least one analgesic comprises of the Capsaicin that is in the range from about 0.001 percent to about 8.000 percent of the gel.

6. The electrically stimulated CBD infused pain-relieving patch that is placed on a human's skin that is used to relieve pain when it is connected to a controlled electrical current generating device of claim 5, the gel comprises of a plurality of inactive ingredients.

7. The electrically stimulated CBD infused pain-relieving patch that is placed on a human's skin that is used to relieve pain when it is connected to a controlled electrical current generating device of claim 1, the gel comprises of a plurality of inactive ingredients.
